(19) United States
(12) Patent Application Publication
(10) Pub. No.: US 2021/0322783 A1
(43) Pub. Date: Oct. 21, 2021

(54) PHOTOTHERAPY DEVICE

(71) Applicant: OLYMPUS CORPORATION, Tokyo (JP)

(72) Inventor: Miho KOJIMA, Tokyo (JP)

(73) Assignee: OLYMPUS CORPORATION, Tokyo (JP)

(21) Appl. No.: 17/365,348

(22) Filed: Jul. 1, 2021

Related U.S. Application Data
(63) Continuation of application No. PCT/JP2019/000470, filed on Jan. 10, 2019.

(51) Int. Cl. A61N 5/06

(52) U.S. Cl. CPC A61N 5/0603; A61N 2005/0626; A61N 2005/063; A61N 2005/061

(57) ABSTRACT

A phototherapy device includes: a tubular first sheath having a transparent portion transmitting light; a tubular second sheath disposed in the first sheath, formed of a light-impermeable material, and having a light transmission region in a portion of a side wall thereof; a tubular third sheath disposed in the second sheath, formed of a light-impermeable material, and having a light transmission region in a portion of a side wall thereof; and an optical fiber passing through insides of the second and third sheaths in a longitudinal direction and emitting light in radial directions of the second and third sheaths, wherein the second and third sheaths are configured to perform at least one of relative rotation and relative movement, and wherein the light transmission regions of the second and third sheaths are configured to be overlapped by means of the relative rotation or the relative movement.

### CROSS-REFERENCE TO RELATED APPLICATIONS

This is a continuation of International Application PCT/JP2019/000470 which is hereby incorporated by reference herein in its entirety.

### TECHNICAL FIELD

The present invention relates to a phototherapy device.

### BACKGROUND ART

In the related art, there is a known device for treating a disease, such as cancer, in a body cavity by using photodynamic therapy (for example, see Patent Literature 1). Photodynamic therapy is a method for treating a lesion site by means of a photochemical reaction of medicine by radiating light onto the lesion site where the medicine has accumulated. The device described in Patent Literature 1 includes an optical fiber and a balloon that covers a distal end portion of the optical fiber and that is inflated in the body cavity. Light is emitted radially from the optical fiber inside the balloon inflated in the body cavity, and the light is radiated onto the cavity wall through the balloon. The balloon serves to uniformize the light radiated onto the cavity wall.

### Citation List

### Patent Literature

{PTL 1} U.S. Pat. No. 6,364,874, Specification

### SUMMARY OF INVENTION

An aspect of the present invention is a phototherapy device including: a tubular first sheath having a transparent portion that transmits light; a tubular second sheath that is disposed in the first sheath and that is formed of a light-impermeable material, the second sheath having a light transmission region that transmits light in a portion of a side wall of the second sheath; a tubular third sheath that is disposed in the second sheath and that is formed of a light-impermeable material, the third sheath having a light transmission region that transmits light in a portion of a side wall of the third sheath; and an optical fiber that passes through insides of the second sheath and the third sheath in a longitudinal direction and that emits light in radial directions of the second sheath and the third sheath, wherein the second sheath and the third sheath are configured to perform at least one of relative rotation about a longitudinal axis and relative movement in a direction along the longitudinal axis, and wherein the light transmission region of the third sheath and the light transmission region of the second sheath are configured to be overlapped by means of the relative rotation or the relative movement.

Another aspect of the present invention is a phototherapy device including: a sheath having a tubular sheath body that is formed of a light-impermeable material and a light-transmissive balloon that is provided at a distal end portion of the sheath body; and an optical fiber that passes through the inside of the sheath body, a distal end portion of which is disposed in the balloon, and that emits light in a radial direction of the sheath body, wherein the sheath body has a light transmission region that transmits light in a portion of a side wall of the sheath body.

### BRIEF DESCRIPTION OF DRAWINGS

FIG. 1 is a side view showing the overall configuration of a phototherapy device according to an embodiment of the present invention.

FIG. 2A is a side view of an outer light-blocking sheath and an inner light-blocking sheath of the phototherapy device in FIG. 1.

FIG. 2B is a side view showing a state in which the inner light-blocking sheath is disposed inside the outer light-blocking sheath.

FIG. 3 is a diagram for explaining the positional relationship between a window in the outer light-blocking sheath and a window in the inner light-blocking sheath.

FIG. 4 is a diagram showing an example of an indicator provided on a stopper.

FIG. 5A is a diagram for explaining a method for using the phototherapy device in FIG. 1.

FIG. 5B is a diagram for explaining the method for using the phototherapy device in FIG. 1.

FIG. 5C is a diagram for explaining the method for using the phototherapy device in FIG. 1.

FIG. 5D is a diagram for explaining the method for using the phototherapy device in FIG. 1.

FIG. 5E is a diagram for explaining the method for using the phototherapy device in FIG. 1.

FIG. 5F is a diagram for explaining the method for using the phototherapy device in FIG. 1.

FIG. 5G is a diagram for explaining the method for using the phototherapy device in FIG. 1.

FIG. 5H is a diagram for explaining the method for using the phototherapy device in FIG. 1.

FIG. 6A is a diagram showing an example configuration of indicators provided on the outer light-blocking sheath and the inner light-blocking sheath.

FIG. 6B is a diagram showing another example configuration of the indicators provided on the outer light-blocking sheath and the inner light-blocking sheath.

FIG. 7 is a side view showing an overall configuration of a modification of the phototherapy device in FIG. 1.

### DESCRIPTION OF EMBODIMENT

A phototherapy device 1 according to an embodiment of the present invention will be described below with reference to the drawings.

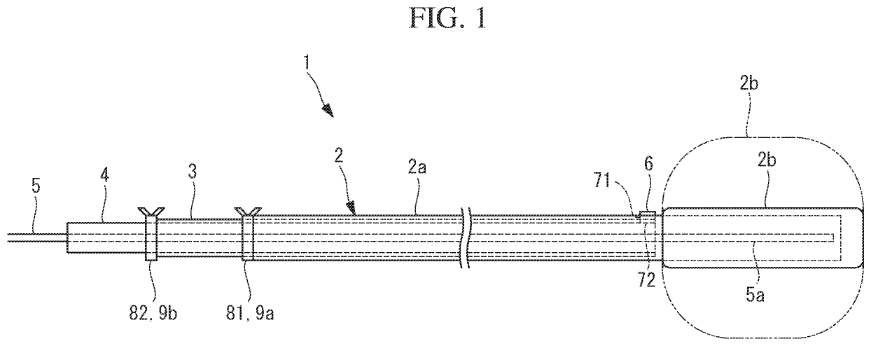

The phototherapy device 1 according to this embodiment is intended for treatment of the bladder and the urethra. As shown in FIG. 1, the phototherapy device 1 includes: a transparent sheath 2 that has a sheath body (first sheath) 2a and a balloon 2b provided at a distal end portion of the sheath body 2a; an outer light-blocking sheath (second sheath) 3 that is inserted into the transparent sheath 2; an inner light-blocking sheath (third sheath) 4 that is inserted into the outer light-blocking sheath 3; an optical fiber (phototherapy member) 5 that is inserted into the inner light-blocking sheath 4 and that emits therapeutic light in the radial direction of the light-blocking sheaths 3, 4; a pressure-sensitive sensor 6; proximity sensors 71, 72; and stoppers 81, 82.

The sheath body 2a and the light-blocking sheaths 3, 4 are elongated tubular members and have flexibility so as to be bendable along the shape of the urethra B. When the phototherapy device 1 is used, the sheath body 2a and the light-blocking sheaths 3, 4 are arranged in a substantially coaxial manner, and a distal end portion 5a of the optical fiber 5 passing through the inside of the inner light-blocking sheath 4 is disposed in the balloon 2b. The inner diameter of the sheath body 2a is larger than the outer diameter of the outer light-blocking sheath 3, and the outer light-blocking sheath 3 inside the sheath body 2a can rotate about a longitudinal axis and can move in a direction along the longitudinal axis with respect to the sheath body 2a. The inner diameter of the outer light-blocking sheath 3 is larger than the outer diameter of the inner light-blocking sheath 4, and the inner light-blocking sheath 4 inside the outer light-blocking sheath 3 can rotate about a longitudinal axis and can move in a direction along the longitudinal axis with respect to the outer light-blocking sheath 3.

The sheath body 2a is formed of a light-transmissive material, and the entire sheath body 2a is a transparent portion that transmits light. The balloon 2b covers the distal end portion of the sheath body 2a, and the interior of the sheath body 2a and the interior of the balloon 2b communicate with each other. The balloon 2b is formed of an elastic material having light transmissivity and, as indicated by a two-dot chain line in FIG. 1, is capable of inflating while deforming along the shape of the inner wall of the bladder, as a result of a fluid being supplied to the interior thereof. The fluid is, for example, air or a liquid such as water. The balloon 2b is light-diffusing and serves to diffuse the therapeutic light emitted from the distal end portion 5a of the optical fiber 5 in the balloon 2b, thereby uniformizing the intensity of the therapeutic light radiated onto the cavity wall. In addition, the position of the sheath body 2a is fixed while the balloon 2b is inflated.

The optical fiber 5 is a side-emission type that radially emits therapeutic light, in lateral directions, from the side surface thereof. The proximal end of the optical fiber 5 is connected to a light source (not shown), and therapeutic light L is supplied from the light source to the optical fiber 5. The optical fiber 5 may emit the therapeutic light from the distal end surface in addition to the side surface so that an area in front of the optical fiber 5 can also be irradiated with the therapeutic light. The optical fiber 5 may emit light from the side surface over the entire length thereof, or may be configured so as to emit the therapeutic light only at a distal-end side portion disposed in the bladder and the urethra.

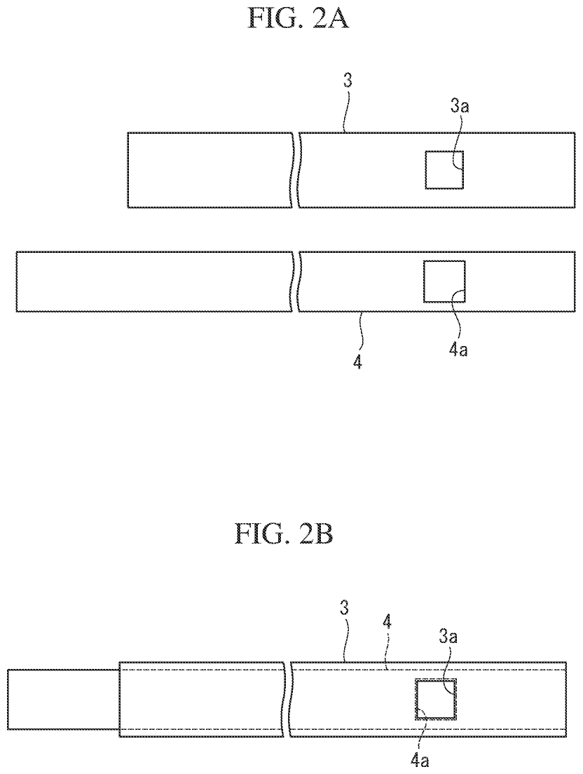

As shown in FIG. 2A, the outer light-blocking sheath 3 has a window (light transmission region) 3a in a portion of the side wall in the circumferential direction and the longitudinal direction thereof. The window 3a is composed of an opening penetrating the side wall in the radial direction or an optically transparent member, and allows light to pass therethrough from the inside to the outside of the outer light-blocking sheath 3. The outer light-blocking sheath 3 is formed of a light-impermeable material, and the entire outer light-blocking sheath 3, excluding the window 3a, has light-blocking properties. The window 3a preferably has a size corresponding to the size of a lesion site, such as a tumor, formed in the urethra.

As shown in FIG. 2A, the inner light-blocking sheath 4 has a window (light transmission region) 4a in a portion of the side wall in the circumferential direction and the longitudinal direction thereof. The window 4a is composed of an opening penetrating the side wall in the radial direction or an optically transparent member, and allows light to pass therethrough from the inside to the outside of the inner light-blocking sheath 4. The inner light-blocking sheath 4 is formed of a light-impermeable material, and the entire inner light-blocking sheath 4, excluding the window 4a, has light-blocking properties.

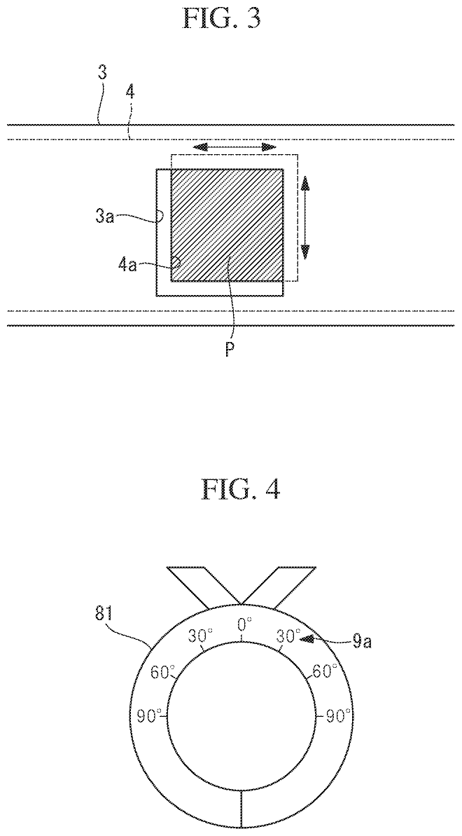

As shown in FIG. 3, the window 4a can be disposed at a position where the window 4a overlaps the window 3a by rotating the inner light-blocking sheath 4 about the longitudinal axis or moving the inner light-blocking sheath 4 in a direction along the longitudinal axis in the outer light-blocking sheath 3. When the window 4a overlaps at least a portion of the window 3a, the therapeutic light can be radiated onto biological tissue outside the outer light-blocking sheath 3 from the optical fiber 5 inside the inner light-blocking sheath 4 through an overlapping region P (see the hatching region in FIG. 3) between the window 4a and the window 3a. At this time, because a portion of the outer light-blocking sheath 3 other than the window 3a has light-blocking properties, the irradiation region of the therapeutic light on the biological tissue is limited to a region facing the overlapping region P.

As a result of the light-blocking sheaths 3, 4 being relatively moved in the longitudinal direction and being relatively rotated about the longitudinal axis, the windows 3a, 4a relatively move in the longitudinal direction and the circumferential direction, and the area of the overlapping region P between the windows 3a, 4a continuously changes. Therefore, by advancing/retracting or rotating the inner light-blocking sheath 4 with respect to the outer light-blocking sheath 3, it is possible to switch between emission and non-emission of the therapeutic light from the window 3a, and in addition, it is possible to change the area of the irradiation region of the therapeutic light on the biological tissue. In particular, it is possible to easily perform fine adjustment of the areas of the overlapping region P and the irradiation region by relatively moving the windows 3a, 4a in the two directions.

The pressure-sensitive sensor 6 is fixed to the outer surface of the sheath body 2a at a position closer to the proximal end than the balloon 2b is. The position at which the pressure-sensitive sensor 6 is fixed to the sheath body 2a is determined according to the size of the bladder, and the pressure-sensitive sensor 6 is configured to be disposed at the boundary between the urethra and the bladder in a state in which the distal end of the transparent sheath 2 is disposed in the vicinity of the inner wall of the bladder. The pressure-sensitive sensor 6 senses a contact pressure against biological tissue. In the process in which the transparent sheath 2 is inserted into the urethra and the bladder, the pressure-sensitive sensor 6 receives a pressure due to contact with the cavity wall in the narrow urethra, and the pressure is released when the pressure-sensitive sensor 6 reaches the wide bladder beyond the urethra. Therefore, a user can recognize that the pressure-sensitive sensor 6 has reached the bladder and the entire balloon 2b is disposed in the bladder, on the basis of a decrease in the contact pressure sensed by the pressure-sensitive sensor 6.

The first proximity sensor 71 is fixed to the sheath body 2a in the vicinity of the pressure-sensitive sensor 6. The second proximity sensor 72 is fixed to the distal end or the vicinity of the distal end of the outer light-blocking sheath 3. The proximity sensors 71, 72 do not respond when the sensors are separated from each other and respond only when the sensors have approached each other. Therefore, in the process of inserting the outer light-blocking sheath 3 into the sheath body 2a, the user can recognize that the distal end of the outer light-blocking sheath 3 has reached the vicinity of the pressure-sensitive sensor 6 on the transparent sheath 2, on the basis of the responses of the proximity sensors 71, 72.

The proximity sensor 72 may be fixed to the distal end or the vicinity of the distal end of the inner light-blocking sheath 4, instead of the outer light-blocking sheath 3.

The stopper (first stopper) 81 is a clip that is attachable to and detachable from the outer circumferential surface of the outer light-blocking sheath 3 outside the sheath body 2a. The stopper 81 attached to the outer circumferential surface of the outer light-blocking sheath 3 abuts against the proximal end surface of the sheath body 2a, thereby preventing movement in a direction in which the outer light-blocking sheath 3 is inserted into the sheath body 2a, while allowing rotation of the outer light-blocking sheath 3 with respect to the sheath body 2a.

The stopper (second stopper) 82 is a clip that is attachable to and detachable from the outer circumferential surface of the inner light-blocking sheath 4 outside the outer light-blocking sheath 3. The stopper 82 attached to the outer circumferential surface of the inner light-blocking sheath 4 abuts against the proximal end surface of the outer light-blocking sheath 3, thereby preventing movement in a direction in which the inner light-blocking sheath 4 is inserted into the outer light-blocking sheath 3, while allowing rotation of the inner light-blocking sheath 4 with respect to the outer light-blocking sheath 3.

The stoppers 81, 82 are respectively provided with indicators 9a, 9b that indicate a relative angle between the window 3a and the window 4a about the longitudinal axes of the light-blocking sheaths 3, 4. For example, as shown in FIG. 4, the indicator 9a of the stopper 81 is a scale indicating a rotation angle about the longitudinal axis. Similarly, the indicator 9b of the stopper 82 is a scale indicating a rotation angle about the longitudinal axis. The user can recognize the relative angle between the two windows 3a, 4a in the body on the basis of the indicators 9a, 9b of the two stoppers 81, 82 outside the body, and can adjust the area of the overlapping region P between the windows 3a, 4a to a desired area.

Next, a method for treating the bladder and the urethra using the phototherapy device 1 will be described.

Prior to the treatment using the phototherapy device 1, the size of the bladder of a patient and the position and size of a lesion site (for example, a tumor) in the urethra are confirmed by a preoperative examination with a CT device or the like. In addition, medicine is administered in advance to the bladder and the lesion site in the urethra. The medicine has the property that it accumulates in the lesion site and exhibits a therapeutic effect on the lesion site through a reaction with therapeutic light.

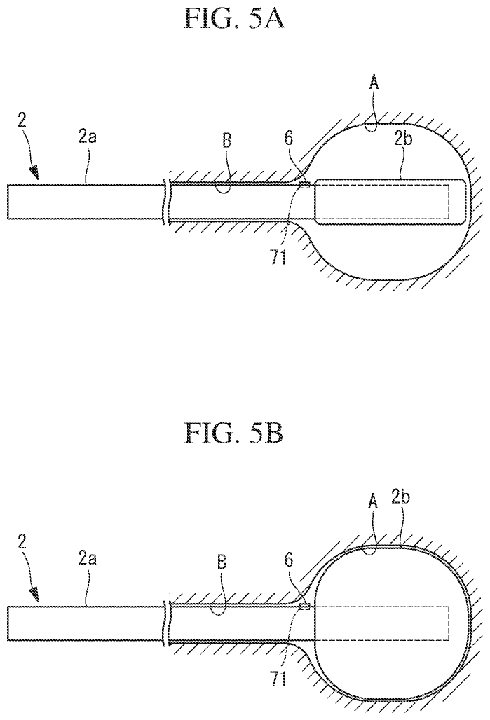

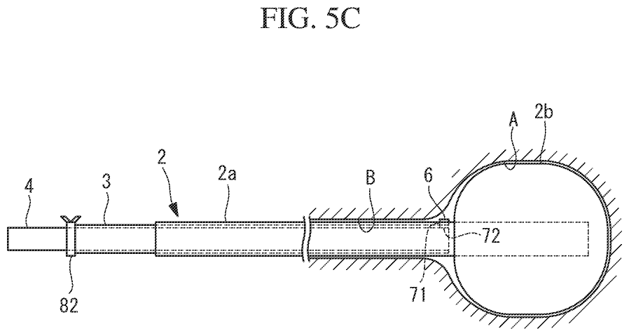

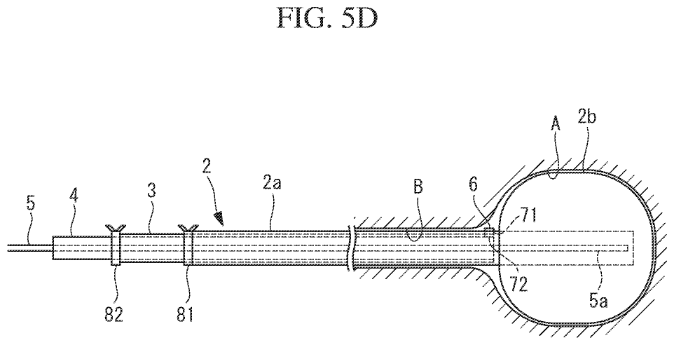

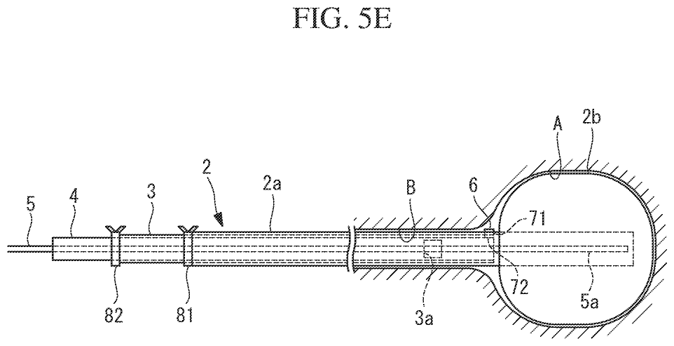

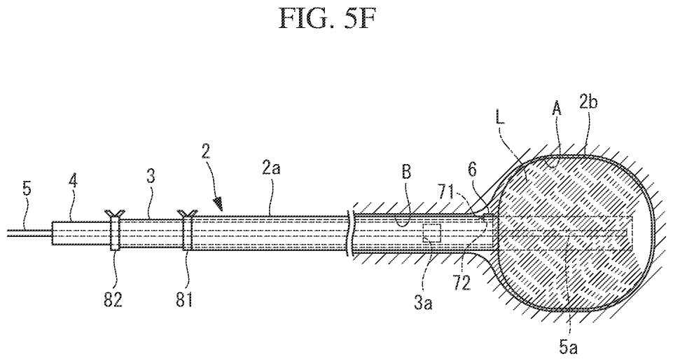

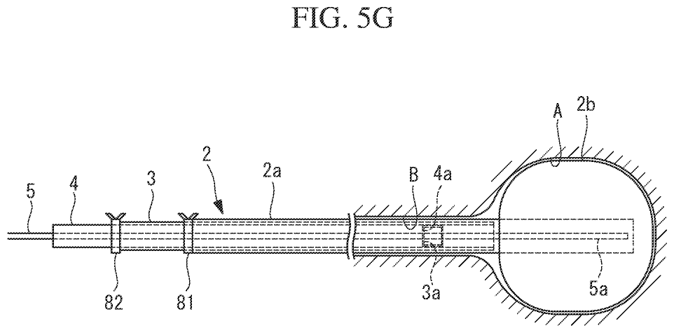

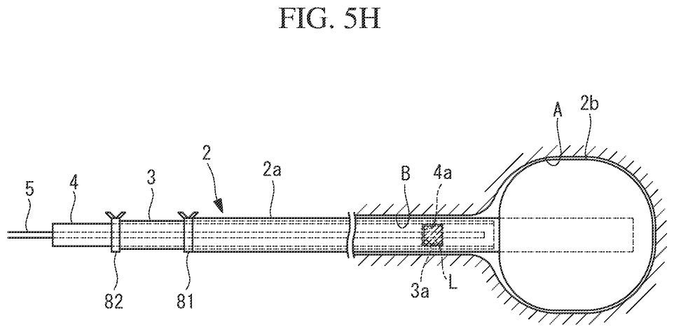

First, as shown in FIG. 5A, the transparent sheath 2 is inserted from the urethral opening to the bladder A through the urethra B. During the insertion of the transparent sheath 2, the user monitors the contact pressure sensed by the pressure-sensitive sensor 6 and stops the transparent sheath 2 when the contact pressure has decreased. By doing so, the pressure-sensitive sensor 6 is disposed at the boundary between the urethra B and the bladder A, and the transparent sheath 2 is located at such a position that the entire balloon 2b is disposed in the bladder A.

Next, as shown in FIG. 5B, the balloon 2b is inflated in the bladder A by supplying a fluid into the balloon 2b. The fluid is supplied, for example, through the sheath body 2a. The transparent sheath 2 is fixed with respect to the urethra B and the bladder A as a result of the inflated balloon 2b being in close contact with the inner wall of the bladder A.

Next, as shown in FIG. 5C, the outer light-blocking sheath 3 and the inner light-blocking sheath 4, which is disposed in the outer light-blocking sheath 3 and to which the stopper 82 is attached, are inserted as a single unit into the sheath body 2a. The light-blocking sheaths 3, 4 are positioned by means of the stopper 82 such that the distal ends of the light-blocking sheaths 3, 4 are arranged at the same position in the longitudinal direction. In this state, the windows 3a, 4a are arranged at the same position in the longitudinal direction. During the insertion of the light-blocking sheaths 3, 4, the user monitors the presence/absence of the responses of the proximity sensors 71, 72 and stops the light-blocking sheaths 3, 4 when the proximity sensors 71, 72 have responded. By doing so, the light-blocking sheaths 3, 4 are located at such a position that the distal ends of the light-blocking sheaths 3, 4 are arranged at the boundary between the urethra B and the bladder A.

Next, as shown in FIG. 5D, the stopper 81 is attached to the outer circumferential surface of the outer light-blocking sheath 3, at a position adjacent to the proximal end surface of the sheath body 2a, and the stopper 81 prevents the light-blocking sheaths 3, 4 from moving in the insertion direction with respect to the sheath body 2a.

Next, the optical fiber 5 is inserted into the bladder A through the inside of the inner light-blocking sheath 4, and the distal end portion 5a is disposed in the balloon 2b. The amount by which the optical fiber 5 is inserted into the inner light-blocking sheath 4 is adjusted, for example, on the basis of the insertion amounts of the light-blocking sheaths 3, 4 from the urethral opening, and the distal end of the optical fiber 5 is disposed at a position closer to the proximal end than the distal end of the sheath body 2a is. Therefore, scales indicating the insertion amounts from the urethral opening may be provided on the outer circumferential surfaces of the light-blocking sheaths 3, 4.

Next, as shown in FIG. 5E, the outer light-blocking sheath 3 is rotated in the sheath body 2a so that the window 3a faces the lesion site. The light-blocking sheaths 3, 4 may be moved in the longitudinal direction in the sheath body 2a according to the position of the lesion site. In addition, the rotation angle of the inner light-blocking sheath 4 is adjusted so that the window 4a does not overlap the window 3a, on the basis of the indicators 9a, 9b of the stoppers 81, 82.

Next, as shown in FIG. 5F, the therapeutic light L is supplied from the light source to the optical fiber 5, and the therapeutic light L is radially emitted from the distal end portion 5a of the optical fiber 5 in the balloon 2b. The therapeutic light L passes through the balloon 2b and is radiated onto the bladder tissue facing the balloon 2b. The medicine in the bladder tissue is activated by the reaction with the therapeutic light L, and the activated medicine exhibits a therapeutic effect on the bladder tissue. At this time, because the light from the optical fiber 5 is blocked by the light-blocking sheaths 3, 4 in the urethra B, the urethral tissue is not irradiated with the light.

Next, as shown in FIG. 5G, the inner light-blocking sheath 4 is rotated in the outer light-blocking sheath 3, the rotation angle of the inner light-blocking sheath 4 is adjusted on the basis of the indicators 9a, 9b of the stoppers 81, 82, and the window 4a is disposed at a position where the window 4a overlaps the window 3a.

Next, as shown in FIG. 5H, the therapeutic light L is supplied from the light source to the optical fiber 5. The therapeutic light L emitted from the optical fiber 5 passes through the overlapping region P between the window 3a and the window 4a and is radiated onto the lesion site in the urethra B facing the overlapping region P. In the lesion site, the medicine is activated by the reaction with the therapeutic light L, and the activated medicine exhibits a therapeutic effect on the lesion site. At this time, the distal end portion 5b of the optical fiber 5 may be stored in the light-blocking sheaths 3, 4 or may be disposed in the balloon 2b.

Subsequently, the balloon 2b is contracted by discharging the fluid from the balloon 2b, and the transparent sheath 2 and the light-blocking sheaths 3, 4 are removed from the bladder A and the urethra B.

As described above, with this embodiment, the therapeutic light L emitted from the optical fiber 5 is radiated onto the urethral tissue only when the two windows 3a, 4a of the light-blocking sheaths 3, 4 disposed in the urethra B overlap each other. Therefore, it is possible to switch between irradiation and non-irradiation of the therapeutic light L on the urethral tissue with a simple operation of rotating or moving the inner light-blocking sheath 4. In addition, it is possible to selectively radiate the therapeutic light L onto the lesion site facing the window 3a, thereby preventing the therapeutic light L from being radiated onto normal tissue around the lesion site.

In addition, by rotating or advancing/retracting the inner light-blocking sheath 4 with respect to the outer light-blocking sheath 3, the irradiation area of the therapeutic light L on the urethral tissue changes. Therefore, it is possible to adjust the irradiation area of the therapeutic light L according to the size of the lesion site, thereby preventing the therapeutic light L from being radiated onto normal tissue around the lesion site in a more reliable manner.

In addition, by rotating and advancing/retracting the outer light-blocking sheath 3 in the transparent sheath 2, it is possible to move the window 3a with respect to the urethra B, and to select the position of the irradiation region of the therapeutic light L. Therefore, it is possible to selectively radiate the therapeutic light L onto lesion sites at various positions in the urethra B.

In this embodiment, instead of or in addition to the indicators 9a, 9b of the stoppers 81, 82, indicators may be provided respectively on the light-blocking sheaths 3, 4. The indicators indicate at least one of the relative angle between the windows 3a, 4a and the relative positions of the windows 3a, 4a in the longitudinal direction, and are provided at proximal end portions of the light-blocking sheaths 3, 4 disposed outside the body when the phototherapy device 1 is used.

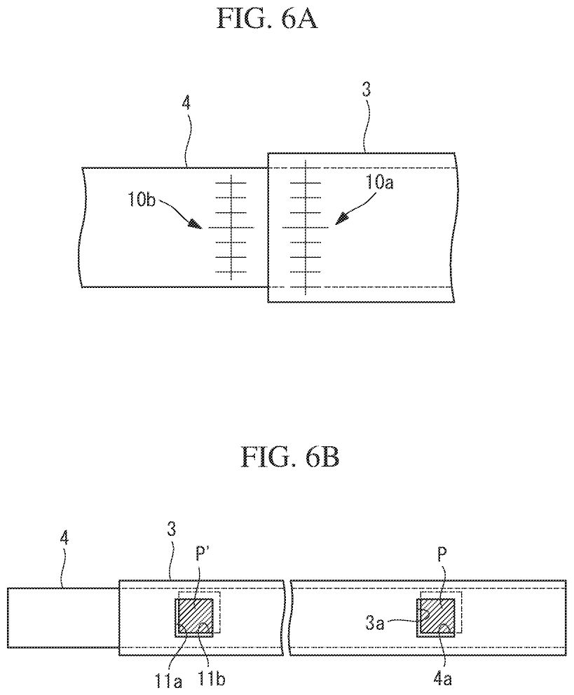

FIGS. 6A and 6B show examples of the indicators of the light-blocking sheaths 3, 4.

Indicators 10a, 10b in FIG. 6A are scales provided on the respective outer circumferential surfaces of the light-blocking sheaths 3, 4. It is possible to recognize the relative angle between the windows 3a, 4a on the basis of the scales 10a, 10b. Scales indicating the relative positions of the windows 3a, 4a in the longitudinal direction may be provided on the outer circumferential surfaces of the light-blocking sheaths 3, 4.

Indicators 11a, 11b in FIG. 6B are windows respectively corresponding to the windows 3a, 4a. The shapes and dimensions of the windows 11a, 11b are the same as those of the windows 3a, 4a, respectively. In addition, the positional relationship between the windows 11a, 11b is the same as the positional relationship between the windows 3a, 4a. In other words, the window 11a is provided at the same position as the window 3a in the circumferential direction of the outer light-blocking sheath 3, and the window 11b is provided at the same position as the window 4a in the circumferential direction of the inner light-blocking sheath 4. In addition, the distance between the windows 3a, 11a in the longitudinal direction of the outer light-blocking sheath 3 and the distance between the windows 4a, 11b in the longitudinal direction of the inner light-blocking sheath 4 are equal to each other. Therefore, the position and area of an overlapping region P′ between the windows 11a, 11b are the same as the position and area of the overlapping region P between the windows 3a, 4a. The user can recognize the relative angle between the windows 3a, 4a and the relative positions of the windows 3a, 4a on the basis of the windows 11a, 11b, and can recognize the position and area of the overlapping region P in a more intuitive manner.

Although the entire sheath body 2a is configured to be a transparent portion in this embodiment, alternatively, a portion of the sheath body 2a may serve as the transparent portion. In this case, the transparent portion is provided in a portion required for irradiating the entire bladder A and the lesion site in the urethra B with the therapeutic light L. Specifically, the transparent portion includes a distal end portion of the sheath body 2a that is disposed in the balloon 2b and a portion on the proximal-end side of the balloon 2b. The transparent portion is preferably provided in a portion from the distal end of the sheath body 2a to a position where the transparent portion covers the window 3a when the phototherapy device 1 is used.

In this embodiment, the inner light-blocking sheath 4 need not necessarily be provided. Because the light-blocking sheath 3 disposed in the urethra B has light-blocking properties, except for the window 3a, in the case in which the inner light-blocking sheath 4 is not provided, the therapeutic light L is emitted only from the window 3a in the urethra. Therefore, it is possible to selectively radiate the therapeutic light L onto the lesion site facing the window 3a, thereby preventing the therapeutic light L from being radiated onto normal tissue around the lesion site. In this case, the therapeutic light L is emitted simultaneously from the balloon 2b and the window 3a, and the bladder A and the lesion site in the urethra B are simultaneously irradiated with the therapeutic light L.

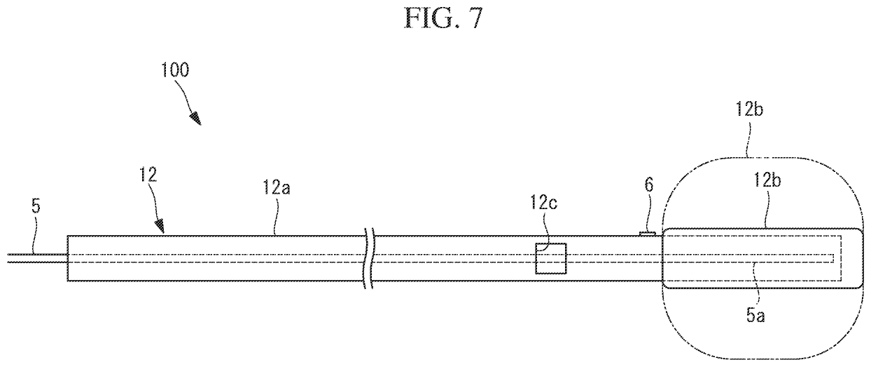

In addition, in this embodiment, a phototherapy device 100 may include a single sheath 12, as shown in FIG. 7, instead of the three sheaths 2, 3, 4. The sheath 12 has a sheath body 12a that is formed of a light-impermeable material and a light-transmissive balloon 12b that is provided at a distal end portion of the sheath body 12a. A window (light transmission region) 12c is formed in a portion of the side wall of the sheath body 12a. Therefore, the bladder A and the lesion site in the urethra B are simultaneously irradiated with the therapeutic light L from the balloon 12b and the window 12c.

Also in the case of using the single sheath 12, it is possible to position the window 12c with respect to the lesion site at any position in the urethra B by rotating and advancing/retracting the sheath body 12a in the urethra B. Therefore, it is possible to selectively radiate the therapeutic light L onto lesion sites at various positions in the urethra B.

### REFERENCE SIGNS LIST

1, 100 phototherapy device transparent sheath
2a sheath body (first sheath)
12a sheath body
2b, 12b balloon
3a, 4a, 12c window (light transmission region)
3 outer light-blocking sheath (second sheath)
4 inner light-blocking sheath (third sheath)
5 optical fiber (phototherapy member)
6 pressure-sensitive sensor
71, 72 proximity sensor
81, 82 stopper
9a, 9b, 10a, 10b, 11a, 11b indicator
12 sheath
A bladder
B urethra
L therapeutic light
P overlapping region

1. A phototherapy device comprising:
a tubular first sheath having a transparent portion that transmits light;
a tubular second sheath that is disposed in the first sheath and that is formed of a light-impermeable material, the second sheath having a light transmission region that transmits light in a portion of a side wall of the second sheath;
a tubular third sheath that is disposed in the second sheath and that is formed of a light-impermeable material, the third sheath having a light transmission region that transmits light in a portion of a side wall of the third sheath; and
an optical fiber that passes through insides of the second sheath and the third sheath in a longitudinal direction and that emits light in radial directions of the second sheath and the third sheath,
wherein the second sheath and the third sheath are configured to perform at least one of relative rotation about a longitudinal axis and relative movement in a direction along the longitudinal axis, and
wherein the light transmission region of the third sheath and the light transmission region of the second sheath are configured to be overlapped by means of the relative rotation or the relative movement.

2. The phototherapy device according to claim 1, wherein the second sheath is configured to perform at least one of rotation about the longitudinal axis and movement in the direction along the longitudinal axis with respect to the first sheath.

3. The phototherapy device according to claim 1, wherein the transparent portion is provided in a portion from a distal end of the first sheath to a position where the transparent portion covers the light transmission region of the second sheath.

4. The phototherapy device according to claim 1, wherein the second sheath and the third sheath have indicators at respective proximal end portions, and the indicators indicate at least one of a relative angle between the light transmission region of the second sheath and the light transmission region of the third sheath about the longitudinal axis and relative positions of the light transmission region of the second sheath and the light transmission region of the third sheath in the direction along the longitudinal axis.

5. The phototherapy device according to claim 2, further comprising:
a first stopper for preventing relative movement of the first sheath and the second sheath in the longitudinal direction; and
a second stopper for preventing relative movement of the second sheath and the third sheath in the longitudinal direction,
wherein the first stopper and the second stopper respectively have indicators, and the indicators indicate a relative angle between the light transmission region of the second sheath and the light transmission region of the third sheath about the longitudinal axis.

6. The phototherapy device according to claim 1, further comprising a pressure-sensitive sensor fixed to the first sheath, wherein the pressure-sensitive sensor senses a contact pressure against biological tissue.

7. The phototherapy device according to claim 1, further comprising:
a first proximity sensor fixed to the first sheath; and
a second proximity sensor fixed to the second sheath or the third sheath,
wherein the first proximity sensor and the second proximity sensor sense that the sensors have approached each other.

8. The phototherapy device according to claim 1, further comprising a light-transmissive balloon that is provided at a distal end portion of the first sheath,
wherein a distal end portion of the optical fiber is disposed in the balloon.

9. A phototherapy device comprising:
a sheath having a tubular sheath body that is formed of a light-impermeable material and a light-transmissive balloon that is provided at a distal end portion of the sheath body; and
an optical fiber that passes through the inside of the sheath body, a distal end portion of which is disposed in the balloon, and that emits light in a radial direction of the sheath body,
wherein the sheath body has a light transmission region that transmits light in a portion of a side wall of the sheath body.
